(19) United States
(12) Patent Application Publication
(10) Pub. No.: US 2010/0119579 A1
(43) Pub. Date: May 13, 2010

(54) DECELLULARIZED LIVER FOR REPAIR OF TISSUE AND TREATMENT OF ORGAN DEFICIENCY

(76) Inventor: Stephen F. Badylak, W. Lafayette, IN (US)

Correspondence Address: BURNS & LEVINSON, LLP, 125 SUMMER STREET, BOSTON, MA, 02110, US

(21) Appl. No.: 12/687,546

(22) Filed: Jan. 14, 2010

Related U.S. Application Data
(63) Continuation of application No. 11/977,645, filed on Oct. 25, 2007, now abandoned.
Application No. 11/977,645 is a continuation of application No. 10/384,213, filed on Mar. 7, 2003, now abandoned.

(51) Int. Cl. A61K 9/00; C12N 5/00; A61K 35/12; A61P 43/00

(52) U.S. Cl. 424/423; 435/325; 424/93.7

(57) ABSTRACT

The present invention provides a liver-derived devitalized mammalian parenchymatous tissue composition which includes an interstitial structure of connective tissue which can serve as a scaffold for tissue repair or regeneration. The devitalized mammalian parenchymatous tissue composition can further include the basement membrane of the tissue.

### TECHNICAL FIELD

This invention relates to devitalized parenchymatous tissue compositions comprising liver, methods of making, and methods of use.

### BACKGROUND OF THE INVENTION

Submucosal tissues of warm-blooded vertebrates are useful in tissue grafting materials. For example, submucosal tissue graft compositions derived from the small intestine have been described in U.S. Pat. No. 4,902,508 (hereinafter the '508 patent) and U.S. Pat. No. 4,956,178 (hereinafter the '178 patent), and submucosal tissue graft compositions derived from urinary bladder have been described in U.S. Pat. No. 5,554,389 (hereinafter the '389 patent). All of these compositions consist essentially of the same tissue layers and are prepared by the same method, the difference being that the starting material is small intestine on the one hand and urinary bladder on the other. The procedure detailed in the '508 patent, incorporated by reference in the '389 patent and the procedure detailed in the '178 patent, includes mechanical abrading steps to remove the inner layers of the tissue, including at least the luminal portion of the tunica mucosa of the intestine or bladder, i.e., the lamina epithelialis mucosa (epithelium) and lamina propria, as detailed in the '178 patent. Abrasion, peeling, or scraping the mucosa delaminates the epithelial cells and their associated basement membrane, and most of the lamina propria, at least to the level of a layer of dense connective tissue, the stratum compactum. Thus, the tissue graft materials previously recognized as soft tissue graft compositions are devoid of epithelial basement membrane.

While tissue graft compositions as described above can be used to create living tissue for tissue replacement, there is still a need for more versatile tissue graft compositions which exhibit mechanical stability similar to that of the host tissue and which can support the growth of a variety of different cell types. To date, selected cell populations such as neurons, blood cells, and endocrine cells are considered to be terminally differentiated and cannot be induced to divide or proliferate further in vivo. These selected cell populations are limited as a source of material for use in graft compositions and the preparation of grafts which support these cells are difficult to make.

### SUMMARY OF THE INVENTION

The present invention provides a liver-derived devitalized mammalian parenchymatous tissue composition that includes an interstitial structure which can serve as a scaffold for tissue repair, restoration, augmentation, or regeneration. The devitalized mammalian parenchymatous liver composition can further include the basement membrane of the liver. For the purposes of this invention, devitalized or acellular means that the cells of the liver have been removed. The presence of the interstitial structure, and optionally also the basement membrane, provide a scaffold which can provide improved in vivo endogenous cell propagation and tissue restoration as compared to matrices derived from the subcutaneous tissue or submucosal tissue of the skin or intestine, respectively. In a preferred embodiment, the invention comprises a devitalized liver that is custom-shaped to conform to a diseased or defective tissue in a patient. The tissue in need of repair, restoration, augmentation, or regeneration includes a target cell type.

The present invention is further based on the finding that the devitalized mammalian parenchymatous liver composition has versatile properties and can serve as a scaffold at a site other than the liver. Moreover, the devitalized mammalian parenchymatous liver composition of the invention supports growth and differentiation of target mammalian cells. Target mammalian cells can include specialized cells which normally do not differentiate or proliferate in vitro, for example, neurons. Examples of other target mammalian cells which may proliferate and differentiate on the mammalian parenchymatous liver composition described herein include, for example, blood cells such as leukocytes, erythrocytes and platelets, stem cells, and endocrine cells such as pancreatic islet cells. Other examples of target mammalian cells include cells which have been genetically altered. The versatile properties of the scaffold of the invention allow the use of this scaffold at different anatomical sites in the body. In combination with appropriate cell types, the scaffold of the invention can further be used to supplement the in vivo production of a biologically active molecule of interest, e.g., a growth factor such as a vascular endothelial cell growth factor (VEGF) or a basic fibroblast growth factor, a hormone such as insulin, or a cytokine such as interleukin-1 The scaffold of the invention can thus serve as an alternative source to produce a biologically active molecule in the body and can be used in the treatment of a disease where there is a need to increase the production of the molecule of interest, e.g., a hormone. The scaffold of the invention can also be used to produce other biologically active molecules for the treatment or prevention of a disease. Such biologically active molecules include antigens, antibodies, enzymes, clotting factors, transport proteins, receptors, regulatory proteins, structural proteins, transcription factors, ribozymes or anti-sense RNA. The scaffold of the invention can further be used to deliver pharmaceutical agents such as antibiotics, anticoagulants such as heparin, and viral inhibitors.

In one aspect of the invention, the invention features a scaffold for promoting extramedullary hematopoiesis in a patient comprising at least a portion of a devitalized mammalian parenchymatous liver in combination with mammalian hematopoietic stem cells. The devitalized tissue can be from an allogeneic tissue source, an autogeneic tissue source or an xenogeneic tissue source. The stem cells can be seeded within the devitalized mammalian parenchymatous liver tissue. The stem cells can be autogeneic, allogeneic or xenogeneic.

In another aspect of the invention, the invention features a scaffold for treatment of an endocrine disorder in a patient comprising at least a portion of a liver-derived devitalized mammalian parenchymatous tissue combined with mammalian endocrine cells. The mammalian endocrine cells can comprise stem cells, pancreatic islet cells, thyroid cells, pituitary cells, or adrenal gland cells and may be allogeneic, autogeneic, or xenogeneic. The devitalized tissue can be allogeneic, autogeneic or xenogeneic.

The present invention further includes a method for the treatment of an endocrine disorder in a patient, e.g., diabetes mellitus, which includes the step of providing a scaffold comprising at least a portion of a devitalized parenchymatous mammalian liver combined with mammalian endocrine cells. The method further includes implanting the scaffold in a patient at an anatomical site other than the site of origin of the devitalized parenchymatous mammalian tissue. Examples of sites where the scaffold can be implanted in a patient include the abdominal cavity, thoracic cavity, bone marrow, intrathecal, subcutaneous tissue, or an intramuscular location.

As used herein, the term “allogeneic tissue” or “allogeneic cell” refers to a tissue or cell which is isolated from an individual and used in another individual of the same species. The term “xenogeneic tissue” or “xenogeneic cell” refers to a tissue or cell which is isolated from an individual of one species and placed in an individual of another species. The term “autogeneic tissue” or “autogeneic cell” refers to a tissue or cell which is isolated from an individual and grafted back into that individual.

### DETAILED DESCRIPTION OF THE INVENTION

The invention is based on the finding that a liver-derived devitalized parenchymatous mammalian tissue, or a portion thereof, can be used as a three dimensional support structure or scaffold according to the invention to augment, repair, restore, or replace a diseased, damaged, missing, or otherwise compromised tissue or organ in the body of a patient. As used herein, restoration shall mean restoring the function of a tissue or restoring the structure of a tissue. The scaffold, in combination with target cells, may be used in vivo to replace or supplement the production of a biologically active molecule of interest. The term parenchymatous refers to tissue found in solid organs. The term “devitalized parenchymatous mammalian liver” refers to the three dimensional support structure which remains when the entire, or substantially entire, parenchymal tissue including the parenchymal cells are removed from the tissue. The three dimensional support system remaining after removing the parenchymal and interstitial cells consists of the extracellular matrix (ECM) and is largely devoid of nuclear and cellular content. The ECM is made up of mostly fibrillar and non-fibrillar collagens. This ECM is referred to herein as the scaffold. The ECM of the scaffold of the invention can be used to grow cells upon and/or within the scaffold. The scaffold however does not only provide a specialized substrate upon which cells can grow upon and within, it also provides specific molecules of interest associated with the substrate. In one embodiment, the ECM of the scaffold may include the basement membrane, which is made up of mostly type IV collagen, laminins and proteoglycans. The ECM provides a supportive framework and microenvironment that allows cells in vitro, whether from a source exogenous to the patient or the patient's own cells, or in vivo, when implanted in a patient's body, to attach, grow and differentiate on the scaffold. As used herein, the term “devitalized mammalian parenchymatous liver” refers to at least a portion of the devitalized mammalian parenchymatous liver or may refer to the whole liver.

Sources of Liver

The liver organ from which the devitalized parenchymatous tissue is derived can be isolated from the patient, from a tissue bank, a human cadaver or from an animal. Useful animals from which a liver can be harvested include animals raised for meat production, including but not limited to pigs, cattle and sheep. Other warm-blooded vertebrates are also useful as a source of liver organs, but the greater availability of such liver organs from animals used for meat production is an inexpensive commercial source of tissue for use in preparation of the devitalized parenchymatous mammalian tissue scaffold according to the invention. In certain incidences it may be preferred to use livers isolated from specially bred or genetically engineered strains of certain species. For example, pigs that are genetically engineered to be free of the galacatosyl, alpha 1,3 galactose (GAL epitope) may be used as the source of tissues for production of the scaffold. Alternatively, pigs from herds that are raised to be free of specific pathogens may be used as a liver source. Mammalian liver used for production of the scaffold composition of the invention may be harvested from an animal of any age group, including embryonic tissues, or market weight pigs, any gender or any stage of sexual maturity.

The devitalized parenchymatous mammalian liver can be obtained from a tissue source which is autogeneic, allogeneic or xenogeneic. According to one embodiment, cells seeded into or onto the devitalized parenchymatous mammalian liver scaffold may be obtained from an autogeneic, allogeneic or xenogeneic source. Exogeneously sourced primary cells, cultured cells, including but not limited to cells from an immortalized cell line, for example, may be introduced into or onto the devitalized acellular parenchymatous mammalian liver scaffold. The scaffold with the exogenous cells or, alternatively, without the cells, may be implanted into a recipient patient's liver or may be implanted at a site remote from the liver.

Decellularization of the Liver

According to the present invention, the liver, or a portion thereof, is prepared by removing the liver, or portion thereof, from a warm-blooded vertebrate, for example, from a patient or from an animal source, for example, a pig. The isolated liver is devitalized by removing the cellular content of the tissue. In one embodiment, the isolated liver is decellularized by treating the tissue with, for example, 0.01% to 5.00% peractic acid, preferably, 0.1% peracetic acid, and subsequently rinsing the tissue with buffered saline and distilled water. The tissue remaining after this treatment is the interstitial structure and the basement membrane. In another embodiment, the basement membrane is also optionally removed by further treating the tissue with specific collagenases (such as collagenese specific for Type IV collagen) to remove the basement membrane. The decellularized state of the resulting scaffold is verified by testing the scaffold for DNA content.

In one embodiment according to the invention, the devitalized mammalian parenchymatous liver scaffold is stored in a frozen and hydrated state. Alternatively, the devitalized mammalian parenchymatous liver scaffold is air dried at room temperature, and then stored. In yet another embodiment, the devitalized mammalian parenchymatous liver scaffold is lyophilized and stored in a dehydrated state at either room temperature or frozen. In yet another embodiment, the devitalized mammalian parenchymatous liver scaffold can be minced and fluidized by digesting the material in proteases, for example pepsin or trypsin, for periods of time sufficient to solubilize the tissue and form a substantially homogeneous solution. The viscosity of the solubilized material can be varied by adjusting the pH to create a gel, gel-sol, or completely liquid state.

In still another embodiment, the present invention contemplates the use of powder forms of the devitalized mammalian parenchymatous liver scaffold. In one embodiment, a powder form of the devitalized mammalian parenchymatous liver scaffold is created by mincing or crushing the devitalized mammalian parenchymatous liver scaffold material to produce particles ranging in size from 0.005 mm² to 2.0 mm². The material is frozen for example, in liquid nitrogen, to perform the crushing procedure. Alternatively, the material is dehydrated to perform the crushing procedure. The crushed form of the material is then lyophilized to form a substantially anhydrous particulate of the devitalized mammalian parenchymatous tissue scaffold. The particulate or powdered form may be compressed together to form a compressed particulate scaffold that may be implanted in a patient's body. In one embodiment according to the invention, cells may be added to the compressed powder or compressed particulate scaffold before the scaffold is implanted in the patient.

The devitalized parenchymatous liver scaffold, in any of a number of its solid, particularized, or fluidized forms, can be used as a scaffold for organ or tissue repair. The devitalized mammalian parenchymatous liver composition of the invention can be sutured into place in its solid sheet form, placed in wounds or body locations in a gel form, or injected or applied in its liquid or particulate form.

Use of the Devitalized Liver

The devitalized mammalian parenchymatous liver scaffold forms a three dimensional support structure that can serve to replace, restore or augment a diseased or damaged tissue. The devitalized liver of the invention is a versatile support structure that can serve as a three dimensional support structure at a remote site in the body. A remote site is an anatomical site other than the liver or a site other than the anatomical site in need of replacement, repair, restoration, or augmentation. For example, the scaffold of the invention is implanted at an anatomical site adjacent a diseased, damaged, or missing portion of the patient's kidney to replace, repair, restore or augment the patient's kidney. The scaffold may be prepared from an autogeneic, allogeneic or xenogeneic tissue source.

In a particular embodiment according to the invention, the devitalized parenchymatous liver scaffold may be used as a substrate that supports the growth and proliferation of a variety of exogenous cell types allowing a target population of cells to expand and thrive on the scaffold when the cells combined with the scaffold are implanted into a patient. The target cells may be primary cells, fetal cells, progenitor cells, or cells from an immortalized cell line, for example. The cells may be epithelial, endothelial, hematopoietic, or connective tissue-origin cells, for example. The cells may be derived from an autogeneic, allogeneic, or xenogeneic source.

According to one embodiment of the invention, the cells are contacted with the devitalized parenchymatous liver scaffold of the invention and permitted to proliferate and differentiate, if required, into a primary cell type that is characteristic of the intended tissue undergoing treatment. Contacting the cells with the scaffold includes coating the outside of the scaffold with the cells, introducing the cells into the scaffold, for example, by injecting the cells into the scaffold, or a combination of coating the scaffold and injecting the cells into the scaffold. The scaffold combined with the cells is implanted at an anatomical site in the patient. The anatomical site may be adjacent to the patient's tissue requiring repair, restoration or augmentation, or the anatomical site into which the scaffold with or without exogenous cells is implanted may be an anatomical site in the patient that is remote from the tissue requiring repair, restoration, or augmentation.

The invention further features using the devitalized parenchymatous liver to support the growth and differentiation of specialized cell populations that include endothelial cells, hematopoietic stem cells, pancreatic islet cells, pituitary cells, or thyroid cells.

For example, in another embodiment, the scaffold may support cells such as specialized cells that synthesize a desired cell product, for example, a biologically active, molecule, e.g., a growth factor such as vascular endothelial cell growth factor (VEGF) or basic fibroblast growth factor, a hormone such as insulin, or a cytokine such as interleukin-1, an antigen, an antibody, an enzyme, a clotting factor, a transport protein, a receptor, a regulatory protein, a structural protein, a transcription factor, a ribozyme or an anti-sense RNA. In one embodiment, the cells may be genetically altered to synthesize the desired biologically active molecule. Genetically altered cells or recombinant cells can be prepared by introducing into the target cell an expression vector which includes a DNA sequence which can encode a biologically active molecule of interest, or fragment thereof Examples of mammalian expression vectors include pCDM8 (Seed, B. (1987) Nature 329:840) and pMT2PC (Kaufman et al. (1987) EMBO J. 6:187-195). The person of ordinary skill in the art would be aware of other vectors suitable for expression of the DNA sequence of interest. These are found for example in Sambrook et al. (1989) Molecular Cloning. A Laboratory Manual 2nd., ed., Cold Spring Harbor Laboratory, Cold Spring Harbor Laboratory Press, Cold Spring Harbor, N.Y. The vector can be introduced into the cell using techniques such as calcium phosphate transfection, DEAE-dextran-mediated transfection, cationic lipid-mediated transfection, electroporation, transduction, infection, lipofection, and other techniques such as those found in Sambrook et al. (supra). The genetically altered cells are contacted with the scaffold and allowed to proliferate and differentiate thereupon.

In another embodiment, the target cells can be used to deliver pharmaceutical agents such as antibiotics, anticoagulants such as heparin and viral inhibitors such as TAP-inhibitor ICP47.

The cells described above may be combined with the devitalized parenchymatous liver scaffold and implanted in the patient at an anatomical site such that it may produce and deliver in vivo a biologically active molecule of interest to the patient. The method for culturing such specialized cells in vitro on the scaffold according to the invention includes the steps of introducing the cells onto the scaffold and culturing the cells in vitro under conditions conducive to proliferation of the cells. The making of the tissue scaffold including cells according to the invention advantageously allows the generation of tissue scaffolds having an expanded cell population from an initially small cell population.

In one embodiment according to the invention, the devitalized parenchymatous liver scaffold having an expanded cell population from a source exogenous to the liver scaffold, is implanted in the patient at an anatomical site that is remote from the tissue requiring repair, restoration, or augmentation. The anatomical sites for implanting the scaffold with the cells include, for example, subcutaneous tissue, intrathoracic cavity, intra-abdominal cavity, intrathecal space, intramedullary cavity, intramuscular sites, peritoneal space, or retroperitoneal space.

In one embodiment, the invention includes a devitalized parenchymatous tissue scaffold which is derived from the liver and is seeded with endocrine cells that secrete a hormone of interest. The scaffold is then implanted into a patient's body at a site other than the liver, e.g., in the kidney. In one embodiment, the scaffold is implanted into a body space, e.g., a body cavity that has a good blood supply. For example, in one embodiment according to the invention, the scaffold can be implanted into the abdominal cavity or the thoracic cavity. Alternatively, the scaffold may be implanted in the retroperitoneal space, peritoneal space, subcutaneous tissue, or intramuscular tissue. Alternatively, the scaffold may be implanted into the bone marrow. In this way the scaffold may be used to produce a biologically active molecule of interest at almost any anatomical site within the body.

In one embodiment, the devitalized parenchymatous liver scaffold is used to support the growth and differentiation of endocrine cells such as pancreatic islet cells, pituitary cells, thyroid cells, and adrenal gland cells. The endocrine cells in combination with the tissue scaffold may secrete a hormone of interest, e.g., thyroid-stimulating hormone, follicle-stimulating hormone, thyroxine, calcitonin, androgens, insulin, glucagon, erythropoietin, calcitriol, insulin-like growth factor-1, angiotensinogen, or thrombopoietin. The devitalized parenchymatous tissue scaffold, in combination with cells, according to the invention, can be used to treat an endocrine disorder in a patient, such as a thyroid disorder, a parathyroid disorder, an adrenal disorder, a pituitary disorder, a reproductive disorder, a hematopoetic disorder, or a pancreatic disorder.

In another embodiment, the devitalized parenchymatous liver scaffold is used to support the growth of thyroid cells and the scaffold and cells are introduced into the thyroid. Alternatively, the scaffold is introduced into the body at a remote site, i.e., at an anatomical site other than the liver or at a site other than the thyroid gland, e.g., the scaffold with the cells can be implanted subcutaneously, in the abdominal cavity, thoracic cavity, intramuscularly, in the intrathecal space, or in the bone marrow.

In another embodiment, the devitalized parenchymatous liver scaffold is used to support the growth of cells which have been genetically altered to produce a biologically active molecule. In one example, the devitalized parenchymatous liver scaffold is used to support the growth of cells which have been genetically modified to produce VEGF. The scaffold and cells are introduced into a body site in, or close to, an area affected by ischemic injury so as to stimulate in that area the local production of blood vessels.

The scaffold of the invention can also be used to deliver a biologically active molecule or pharmaceutical agent in a controlled release manner. In one embodiment, the molecule or agent of interest is provided in a polymer and then incorporated into scaffold using crosslinking methods such as carbodiimide, dehydrothermal methods, aldehydes, or photoxidizers. The scaffold of the invention is then introduced into the body and the polymer is so designed that as it degrades, the biologically active molecule or agent is freed and made available to the body. In another embodiment, the bioactive molecule or agent is directly incorporated into the scaffold and introduced into the body. The degradation of the scaffold in the body results in the controlled release of the molecule or agent.

Liver

The liver-derived devitalized parenchymatous tissue scaffold is prepared by obtaining a liver from a warm-blooded vertebrate, for example, a pig. The tissue is decellularized by treating the liver with 0.01% to 5.00% peracetic acid, preferably, 0.1% peracetic acid for about 5 to 120 minutes, preferably, 15 minutes at a temperature of 25° C. to 40° C., preferably, 37° C., and subsequently rinsing with buffered saline and distilled water. The remaining tissue scaffold includes the extracellular matrix and the basement membrane. In one embodiment according to the invention, the basement membrane is removed by further treating the tissue with specific collagenases to remove the basement membrane. The resulting devitalized parenchymatous tissue scaffold is cell free as verified by measuring the DNA content in the scaffold.

The components of the interstitial matrix with or without the basement membrane of the liver provide a scaffold that has superior biologic tissue remodeling properties and provides support and promotes growth of cells introduced into or on the scaffold. The scaffold derived from the liver can thus be used for the replacement, repair, restoration, or augmentation of body tissues and organs. For example, the scaffold derived from the liver can be used to provide support and promote growth of cells such as endothelial cells, hematopoietic cells, islet cells, pituitary cells, thyroid cells, or stem cells. The scaffold combined with these cells can be implanted into an anatomical site within a patient's body. For example, the scaffold onto which thyroid cells have been grown can be introduced into the thyroid. In a preferred embodiment, the scaffold is introduced into the body at a remote site, i.e., at an anatomical site other than the liver or at a site other than the anatomical site in need of replacement, repair, restoration, or augmentation. The scaffold of the liver is thus implanted at a site in the body other than in the liver and other than the thyroid gland, e.g., the scaffold with the cells can be implanted subcutaneously, in the abdominal cavity, thoracic cavity, intramuscularly, intrathecally, or in the bone marrow.

The following examples will serve to better demonstrate the successful practice of the present invention.

### Exemplification

### Example 1

### Liver-Derived Devitalized Parenchymatous Tissue Scaffold: Endothelial Cell and Fibroblast Growth, Proliferation, and Differentiation

The liver of a pig is surgically removed using standard techniques for tissue removal. The liver is decellularized by treating the liver with 0.1% peracetic acid in a bath temperature of 37° F. for a duration of 15 minutes. The bath is continuously agitated by a magnetic stirring mechanism and subsequently the liver is rinsed with buffered saline followed by distilled water. The remaining material consists of the extracellular matrix (ECM) which has a DNA content that is essentially zero (no difference from background readings of an acellular control solution). The scaffold may be used to support the growth of human microvascular endothelial cells and 3T3 fibroblasts in vitro.

### Example 2

### Liver-Derived Devitalized Parenchymatous Tissue Scaffold: Treatment of Diabetes Mellitus

The parenchymatous devitalized tissue scaffold according to the invention can be used to treat an endocrine disorder, e.g., diabetes mellitus. To do this, pancreatic islet cells are obtained, as described in, for example, U.S. Pat. No. 5,695,998, and cultured in vitro on a liver-derived parenchymatous devitalized tissue scaffold according to the invention prepared as described above. The use of autologous pancreatic islet cells is preferred to minimize cell rejection by the patient's (recipient's) immune system. The islet cells are plated onto the surface or, alternatively, injected into the scaffold, and allowed to thrive on the tissue scaffold. The scaffold, in combination with the pancreatic islet cells, is then implanted into the diabetic patient to aid in glucose regulation by appropriate secretion of insulin. In one embodiment, the scaffold in combination with the pancreatic islet cells is sized and shaped to be implanted at a site other than the pancreas, e.g., elsewhere in the abdominal cavity or in the thoracic cavity.

### Example 3

### Liver-Derived Devitalized Parenchymatous Tissue Scaffold: Treatment of Bone Marrow Disease

The scaffold as described herein may be used to culture stem cells. The stem cells may be induced to differentiate into a particular cell type of interest by introducing an appropriate growth factor. The scaffold can thus serve to promote extramedullary hematopoiesis in a patient. The scaffold is seeded with stem cells, e.g., autogeneic stem cells, allogeneic stem cells, or xenogeneic stein cells.

The devitalized parenchymatous liver scaffold is a substrate on which pluripotential stem cells may be cultured for implantation in combination with the liver-derived devitalized parenchymatous tissue scaffold in a patient's body. Pluripotential stem cells include, but are not limited to, hematopoietic stem cells. Hematopoietic stem cells may proliferate and differentiate into any cell type of the white blood cell series, the red blood cell series, megakaryocyte series, or their combination, for example, neutrophils, mature red blood cells, platelets, or their combination, respectively.

In a particular embodiment according to the invention, hematopoietic stem cells are coated on the surface and injected into the liver-derived devitalized parenchymatous tissue scaffold. The devitalized parenchymatous tissue scaffold may be derived from a xenogeneic tissue source, such as a pig. The cells may be in contact with the devitalized parenchymatous liver scaffold for a few minutes to a few days prior to implantation of the devitalized parenchymatous tissue scaffold with the hematopoietic stem cells at an anatomical site in a patient in need of hematopoiesis. In one embodiment, for example, the cells are cultured on the tissue scaffold long enough to permit a portion of the cell population to differentiate into a terminally differentiated blood cell type, for example, a mature leukocyte.

The scaffold with the hematopoietic cells may be sized and shaped to be implanted in the patient's body at anatomical sites including, but not limited to, subcutaneous tissue, the medullary cavity, the thoracic cavity, the abdominal cavity, or injected into the kidney, spleen, or lymph node.

### Example 4

### Liver-Derived Devitalized Parenchymatous Tissue Scaffold: Treatment of Parkinson's Disease

In another embodiment according to the invention, the devitalized parenchymatous liver scaffold is a substrate with which dopamine-producing progenitor cells, mature dopamine-producing cells, or cells genetically altered to produce dopamine are combined for implantation in a patient with Parkinson's Disease. According to the invention, the devitalized parenchymatous tissue scaffold is prepared as described above. In a particular embodiment according to the invention, the dopamine-producing cells are applied to the surface of the devitalized parenchymatous liver scaffold and/or injected into the devitalized parenchymatous liver scaffold. The scaffold with the cells may be implanted at anatomical sites including, but not limited to, intracranial, intrathecal, intrathoracic, intraabdominal or at subcutaneous sites in a patient having Parkinson's Disease.

### Example 5

### Liver-Derived Devitalized Parenchymatous Tissue Scaffold: Treatment of Anemia-Associated with Renal Failure

In another embodiment according to the invention, the devitalized parenchymatous liver scaffold is a substrate with which erythropoietin-producing progenitor cells, mature erythopoietin-producing cells, or cells genetically altered to produce erythropoietin are combined for implantation in a patient having anemia associated with renal disease, for example, a kidney transplant patient. Cells which produce biologically-active molecules which stimulate erythrogenesis other than erythropoietin may also be combined with the devitalized parenchymatous tissue scaffold according to the invention to treat anemic patients.

According to this embodiment of the invention, a devitalized parenchymatous liver scaffold is prepared as described above. The erythropoietin-producing cells may be combined with the devitalized parenchymatous tissue scaffold as described above and implanted in the anemic patient at sites including, but not limited to, intramedullary, intraabdominal, intrathoracic, intracranial, or in the spleen, or kidney.

### Example 6

### Liver-Derived Devitalized Parenchymatous Tissue Scaffold: Augmentation of the Damaged Urinary Bladder

In yet another embodiment according to the invention, the devitalized parenchymatous liver scaffold is a substrate that may be used to repair, replace, restore, or augment damaged tissue. In a particular embodiment, the devitalized parenchymatous tissue scaffold is placed in contact with a damaged portion of the urinary bladder. In one embodiment, the scaffold is combined with urinary bladder epithelial stem cells, mature primary urinary bladder epithelial cells, or cultured urinary bladder epithelial cells. The scaffold combined with the cells is implanted in the patient's body at the anatomical site in need of repair, restoration, regeneration, or augmentation.

We claim:

1. A scaffold for promoting restoration of a tissue when implanted at an anatomical site in a patient, comprising:
at least a portion of a liver-derived devitalized mammalian parenchymatous tissue combined with a target mammalian cell population, wherein the combined tissue and cell population is sized and shaped for implantation in the patient at the anatomical site remote from the tissue requiring restoration.

2. The scaffold of claim 1 wherein the devitalized mammalian liver tissue further comprises a basement membrane.

3-5. (canceled)

6. The scaffold of claim 1 wherein the cell population is a population of stem cells introduced into the tissue.

7. The scaffold of claim 6 wherein the stem cells comprise autogeneic stem cells.

8. The scaffold of claim 6 wherein the stem cells comprise allogeneic stem cells.

9. The scaffold of claim 6 wherein the stem cells comprise xenogeneic stem cells.

10. The scaffold according to claim 1 wherein the tissue undergoing restoration comprises an endocrine tissue.

11. The scaffold of claim 1 wherein the target cell population comprises mammalian endocrine cells.

12. The scaffold of claim 11 wherein the mammalian endocrine cells comprise pancreatic islet cells.

13. The scaffold of claim 11 wherein the mammalian endocrine cells comprise pituitary cells.

14. The scaffold of claim 11 wherein the mammalian endocrine cells comprise thyroid cells.

15. The scaffold of claim 11 wherein the mammalian endocrine cells comprise cells from the adrenal gland.

16-18. (canceled)

19. The scaffold of claim 10 wherein the mammalian endocrine cells are autogeneic.

20. The scaffold of claim 10 wherein the mammalian endocrine cells are allogeneic.

21. The scaffold of claim 10 wherein the mammalian endocrine cells are xenogeneic.

22. A method for promoting restoration of a tissue when implanted at an anatomical site in a patient, comprising:
providing at least a portion of a liver-derived devitalized mammalian parenchymatous tissue combined with a target mammalian cell population, wherein the combined tissue and cell population is sized and shaped for implantation at the anatomical site in the patient; and
implanting the combined tissue and cell population into a site remote from the tissue requiring restoration.

23. The method of claim 22, wherein the scaffold is implanted subcutaneously.

24. The method of claim 22, wherein the scaffold is implanted into the abdominal cavity.

25. The method of claim 22, wherein the scaffold is implanted into the thoracic cavity.

26. The method of claim 22, wherein the scaffold is implanted subcutaneously.
